(19) United States
(12) Patent Application Publication
(10) Pub. No.: US 2013/0079711 A1
(43) Pub. Date: Mar. 28, 2013

(54) ENDOSCOPE MANIPULATION ADAPTER

(71) Applicants: Archana Appukuttan Nair, Eden Prairie, MN (US); Kevin C. Olds, Baltimore, MD (US); Russell H. Taylor, Severna Park, MD (US)

(72) Inventors: Archana Appukuttan Nair, Eden Prairie, MN (US); Kevin C. Olds, Baltimore, MD (US); Russell H. Taylor, Severna Park, MD (US)

(73) Assignee: The Johns Hopkins University, Baltimore, MD (US)

(21) Appl. No.: 13/595,643

(22) Filed: Aug. 27, 2012

Related U.S. Application Data
(60) Provisional application No. 61/527,236, filed on Aug. 25, 2011.

(51) Int. Cl. A61B 1/005

(52) U.S. Cl. CPC A61B 1/0052
USPC 604/95.04

(57) ABSTRACT

An endoscope manipulation adapter includes a support frame having a first end configured to be rigidly fixed relative to a handpiece of a flexible endoscope, and a manipulation assembly arranged at a second end of the support frame. The manipulation assembly defines a channel for at least one of a flexible endoscope shaft or a flexible endoscope insertion component to traverse through in an axial direction to assist in manipulating the at least one of the flexible endoscope shaft or the flexible endoscope insertion component during use of the flexible endoscope.

### CROSS-REFERENCE OF RELATED APPLICATION

This application claims priority to U.S. Provisional Application No. 61/527,236 filed Aug. 25, 2011, the entire content of which is hereby incorporated by reference.

### BACKGROUND

1. Field of Invention

The field of the currently claimed embodiments of this invention relates to endoscopes, and more particularly to endoscope manipulation adapters.

2. Discussion of Related Art

Endoscopy is used for many surgical procedures including laryngeal, trans- oral, and GI tract surgeries. These surgeries utilize either flexible or rigid endoscopes in their procedures. While manipulators of rigid endoscopes are well developed, especially for laparoscopic applications such as the Da Vinci Robot, robotic manipulation of flexible endoscopes is not well developed.

Manual scope manipulation presents significant challenges, particularly when using longer flexible scopes. Bronchoscopy, for example, requires the surgeon to feed 18-24 inches of scope shaft down the patient's airway by hand, forcing the surgeon to repeatedly lift the endoscope over his/her head in order to feed the end into the patient's mouth. The flexibility of the endoscope shaft and large number of insertions and extractions in a typical brochoscopic procedure make this process difficult, time-consuming and physically tiring.

Robotic manipulation of flexible endoscopes is a much more difficult process. Many researchers have addressed this by engineering a fully robotic remotely controlled flexible endoscope, but such solutions are expensive, complex, and present significant regulatory hurdles. One example of a flexible endoscope manipulator was proposed by Suzumori et al (Suzumori et al. New pneumatic rubber actuators to assist colonoscope insertion. Proceedings 2006 IEEE International Conference on Robotics and Automation. ICRA 2006) that uses a pneumatic device to control a colonoscope. Another approach that emphasizes diagnostic use of short flexible endoscopes is the handheld robotic manipulator from Technical University of Munich (R. Eckl, J. D. J. Gumprecht, G. Strauss, M. Hofer, A. Dietz and T. C. Lueth. Comparison of manual Steering and Steering via Joystick of a flexible Rhino Endoscope 32nd Annual International Conference of the IEEE EMBS Buenos Aires, Argentina, Aug. 31-Sep. 4, 2010). This manipulator provides handle manipulation and scope rotation, but relies on the physician to manually feed the scope in and out of the patient (see, FIG. 1). Since this design lacks a translational degree of freedom, it is intended mostly for applications involving short endoscopes, like upper airway viewing.

The Robotic Endolaryngeal Flexible (Robo-ELF) scope system (“Robotic System to Augment Endoscopes,” U.S. application Ser. No. 13/232,617, assigned to the same assignee as the current application, the entire contents of which are incorporated herein by reference) is unique in its design because it uses clinically approved flexible endoscopes but robotically controls all three degrees of freedom (DOF) (Kevin Olds, Alexander Hilel, M. D., Elizabeth Cha, Martin Curry, D. O., Lee Akst, M. D., Jeremy Richmon, M. D., et al. A robotic assistant for trans-oral surgery: The robotic endo-laryngeal flexible (robo-ELF) scope Hamlyn Symposium on Medical Robotics, Imperial College, London 2011). The current design of the Robo-ELF Scope system (see, e.g., FIG. 2) is optimized for a laryngoscope and has limited translational motion (approximately 3-4 inches). Though the robot itself is easily capable of manipulating other flexible endoscope types, this limit on active translational motion makes manipulating longer endoscopes difficult.

Specialized tools are also available for flexible endoscopes that have working channels. The tools can be inserted into the working channel of the endoscope, and emerge from the distal end, allowing surgeons to perform biopsy, ablation, and other surgical tasks, for example (FIG. 3).

Therefore, there remains a need for improved flexible endoscopes and/or adapters for flexible endoscopes.

### SUMMARY

An endoscope manipulation adapter according to an embodiment of the current invention includes a support frame having a first end configured to be rigidly fixed relative to a handpiece of a flexible endoscope, and a manipulation assembly arranged at a second end of the support frame. The manipulation assembly defines a channel for at least one of a flexible endoscope shaft or a flexible endoscope insertion component to traverse through in an axial direction to assist in manipulating the at least one of the flexible endoscope shaft or the flexible endoscope insertion component during use of the flexible endoscope.

An endoscope manipulation adapter according to an embodiment of the current invention includes a support frame having a first end configured to be rigidly fixed relative to a patient or an object to be viewed with a flexible endoscope, and a manipulation assembly arranged at a second end of the support frame. The manipulation assembly defines a channel for a flexible endoscope shaft to traverse through in an axial direction to assist in manipulating the flexible endoscope shaft during use of the flexible endoscope.

### BRIEF DESCRIPTION OF THE DRAWINGS

Further objectives and advantages will become apparent from a consideration of the description, drawings, and examples.

FIG. 1 shows the T.U. Munich handheld robotic scope manipulator which can be used in conjunction with an endoscope manipulation adapter according to an embodiment of the current invention.

FIG. 2 shows an embodiment of the Robo-ELF Scope manipulation system which can be used in conjunction with an endoscope manipulation adapter according to an embodiment of the current invention.

FIG. 3 shows an example of an endoscope with biopsy tool inserted through the working channel which can be used in conjunction with an endoscope manipulation adapter according to an embodiment of the current invention.

FIG. 4 is a schematic illustration of an endoscope manipulation adapter that is a bronchoscope adapter according to an embodiment of the current invention.

FIG. 5 shows an endoscope manipulation adapter according to an embodiment of the current invention.

FIG. 6 shows a portion of the endoscope manipulation adapter of FIG. 5. A) Spring-loaded traction wheel. B) Grooved guide wheel. C) DC Motor which powers wheels

FIG. 7 is a front view of the endoscope manipulation adapter of FIG. 6.

FIG. 8 is a perspective view of the endoscope manipulation adapter of FIG. 6.

FIG. 9 is a schematic illustration of an endoscope manipulation adapter configured for use with the Robo-ELF Scope manipulation system using an active roller mechanism to provide additional translational range according to an embodiment of the current invention.

FIG. 10 is a schematic illustration of an endoscope manipulation adapter configured for use with a handheld scope manipulation robot to provide translational motion according to an embodiment of the current invention.

FIG. 11 is a schematic illustration of an endoscope manipulation adapter to provide an active roller to drive endoscopic tools through the working channel of a flexible endoscope according to an embodiment of the current invention.

FIG. 12 is a schematic illustration of an endoscope manipulation adapter configured for use with a Robo-ELF-like robot with extended translation using fairlead and telescoping tube guide according to an embodiment of the current invention.

FIG. 13 is a schematic illustration of an endoscope manipulation adapter configured for use with a handheld endoscope using active fairlead mechanism according to an embodiment of the current invention.

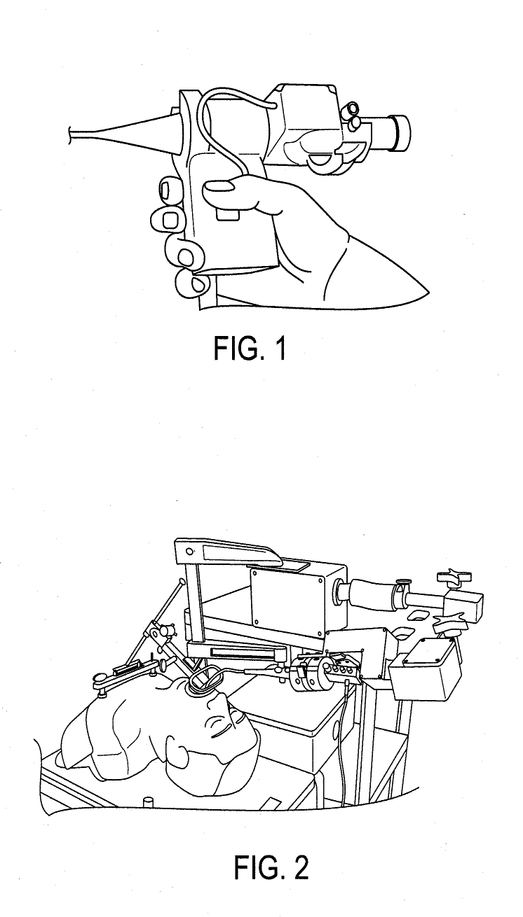

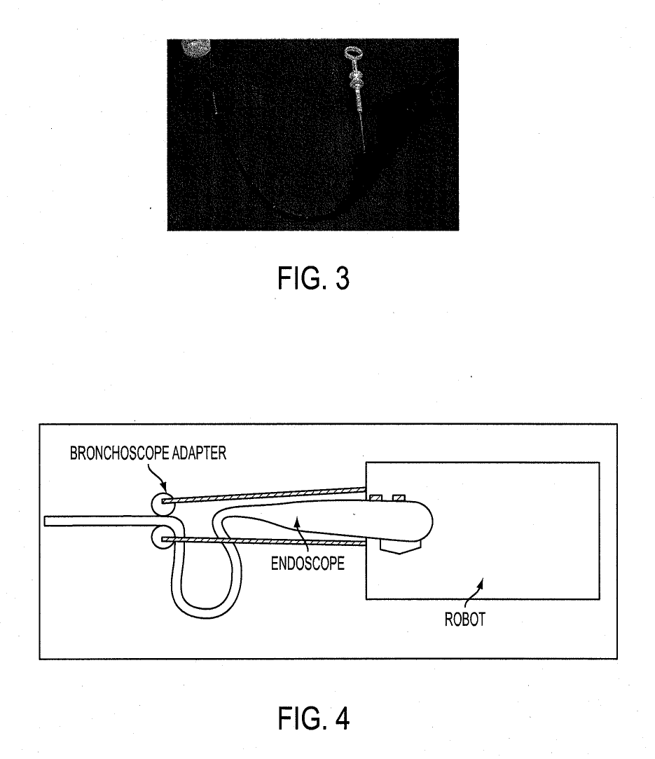

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

Some embodiments of the current invention are directed to endoscope manipulation adapters to feed the scope tip in a smooth and constrained manner without moving the scope body. For example, some embodiments of the current invention can be used in conjunction with a robotic system such that the endoscope manipulation adapter uses the flexibility of the endoscope shaft to manipulate the scope tip insertion, while the scope body is held stationary in the robot. (For example, see FIG. 4.) When the scope is fully out of the patient, the tip of the scope can be held in place with the endoscope manipulation adapter while the extra scope shaft is allowed to hang loosely. The high flexibility and small radius of curvature of modern distal-chip scopes allows the endoscope manipulation adapter to manipulate the scope close to the scope body.

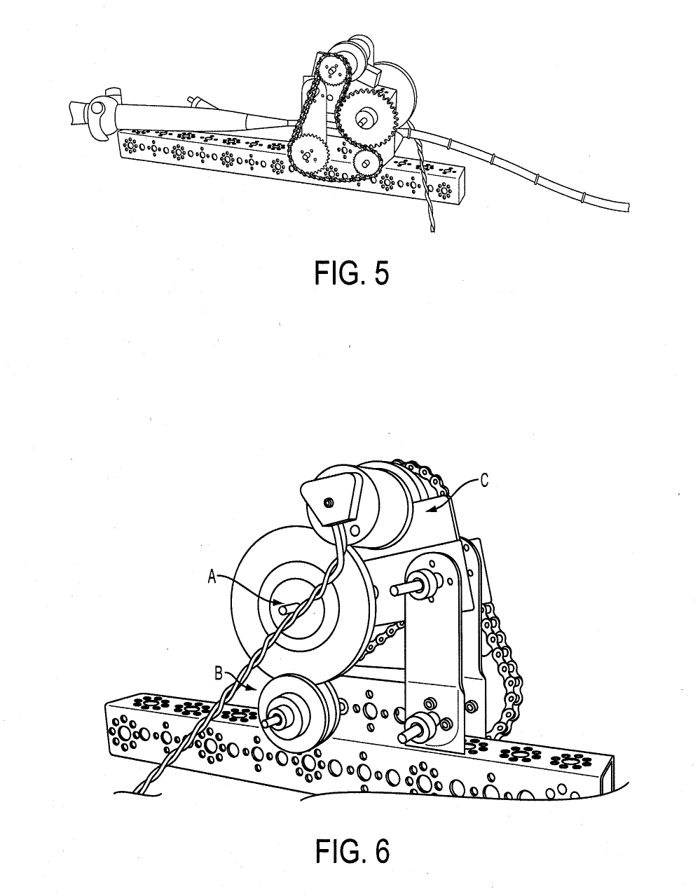

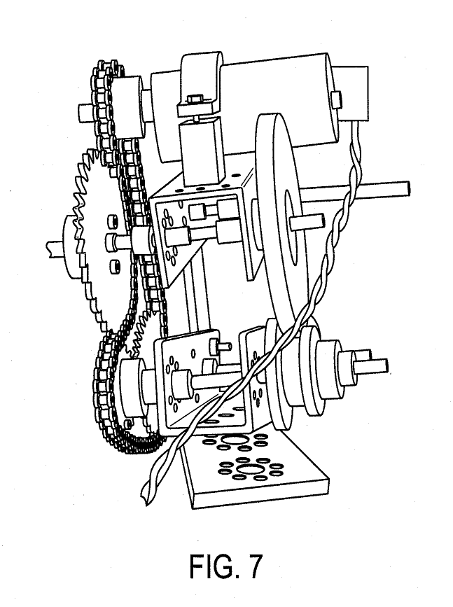

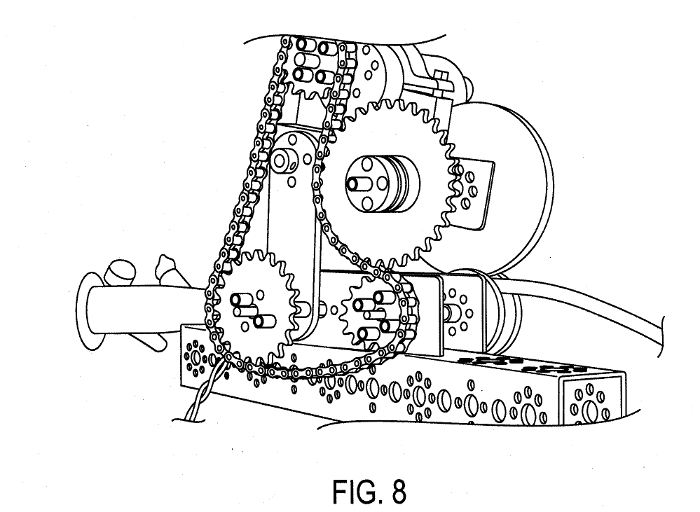

In an embodiment, the adapter design features a double wheel system with the scope shaft moving forward and back between the wheels (FIGS. 5-8). One of the wheels has a groove around its circumference to prevent unwanted sideways motion of the scope, while the other holds the scope shaft in the groove using a tensioning spring. The axles are driven using a serpentine chain sprocket system attached to a DC motor. The design utilizes the flexibility of the endoscope to direct the motion of the scope in and out of the patient.

The following will describe some embodiments using two main approaches, with four different applications for each. However, the general concepts of the current invention are not limited to these particular embodiments. The first approach uses an active roller mechanism to control the endoscope shaft independently of the endoscope body. The second approach uses passive or active wheels, fairleads, and/or other mechanical guides, including but not limited to, telescoping rods or compression spring mechanisms, to direct the scope shaft during insertion.

Each of these approaches could be applied in any or all of the following four different ways: First, to augment a system such as, but not limited to, Robo-ELF; Second, to augment a handheld robotic endoscope manipulation system such as, but not limited to, the T.U. Munich device; Third, to augment use of the endoscope alone with no additional robotic system; and Fourth, to augment flexible tools inserted through the working channel of the endoscope.

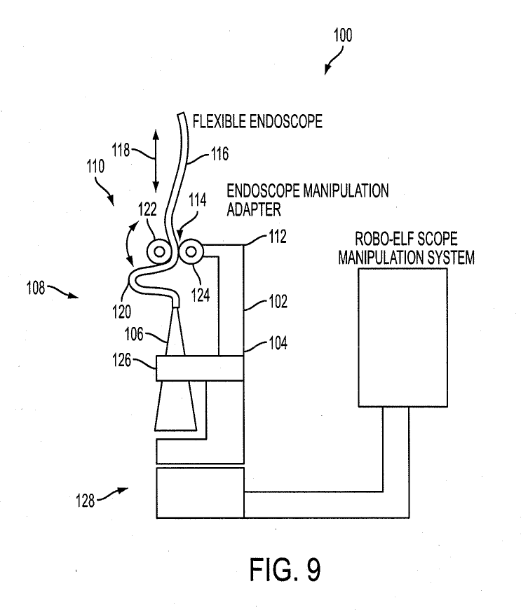

FIG. 9 is a schematic illustration of an endoscope manipulation adapter 100 according to an embodiment of the current invention. The endoscope manipulation adapter 100 has a support frame 102 having a first end 104 configured to be rigidly fixed relative to a handpiece 106 of a flexible endoscope 108. The endoscope manipulation adapter 100 also has a manipulation assembly 110 arranged at a second end 112 of the support frame 102. The manipulation assembly 110 defines a channel 114 for at least one of a flexible endoscope shaft 116 or a flexible endoscope insertion component (not shown in FIG. 9) to traverse through in an axial direction 118 to assist in manipulating the at least one of the flexible endoscope shaft 116 or the flexible endoscope insertion component during use of the flexible endoscope 108.

The support frame 102 and the manipulation assembly 110 reserve space for accommodating slack 120 in the at least one of the flexible endoscope shaft 116 or the flexible endoscope insertion component. The manipulation assembly 110 includes a pair of rollers (122, 124) arranged to define the channel 114. At least one roller 122 or 124 of the pair of rollers (122, 124) is held in a biased position by a spring mechanism to be held against the flexible endoscope shaft or the flexible endoscope insertion component (FIGS. 5-8, for example).

In some embodiments, the manipulation assembly 110 can further include a drive assembly operatively connected to at least one roller of the pair of rollers (122, 124) to actively control the flexible endoscope shaft 116 or the flexible endoscope insertion component. See FIGS. 5-8 for an example of a drive assembly that includes an electric motor and a sprocket and serpentine chain configuration. However, the general concepts of the current invention are not limited to this example. Furthermore, manipulation assembly 110 can be a passive assembly in other embodiments of the current invention.

In some embodiments, the first end 104 of the support frame 102 can be configured to be attached to the handpiece 106 of the flexible endoscope 108. For example, component 126 can be a portion of the first end 104 of the support frame 102. In some further embodiments, as is illustrated in FIG. 9, the support frame 102 can be attached to a robotic system 128. Alternatively, the component 126 can be a portion of the robotic system 128, rather than the endoscope manipulation adapter 100, such that the first end 104 of the support frame 102 is attached to the robotic system 128.

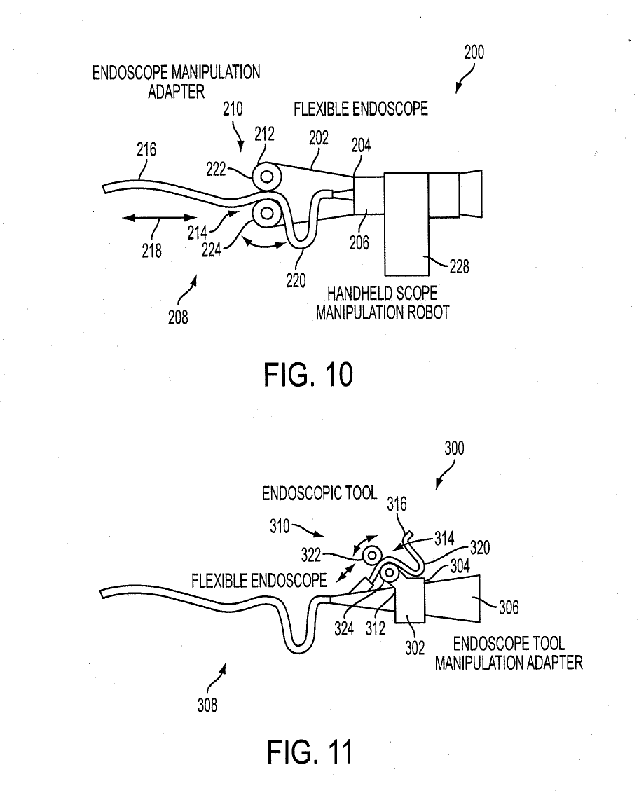

FIG. 10 is a schematic illustration of an endoscope manipulation adapter 200 according to another embodiment of the current invention. The endoscope manipulation adapter 200 has a support frame 202 having a first end 204 configured to be rigidly fixed relative to a handpiece 206 of a flexible endoscope 208. The endoscope manipulation adapter 200 also has a manipulation assembly 210 arranged at a second end 212 of the support frame 202. The manipulation assembly 210 defines a channel 214 for at least one of a flexible endoscope shaft 216 or a flexible endoscope insertion component. (not shown in FIG. 10) to traverse through in an axial direction 218 to assist in manipulating the at least one of the flexible endoscope shaft 216 or the flexible endoscope insertion component during use of the flexible endoscope 208.

The support frame 202 and the manipulation assembly 210 reserve space for accommodating slack 220 in the at least one of the flexible endoscope shaft 216 or the flexible endoscope insertion component. The manipulation assembly 210 includes a pair of rollers (222, 224) arranged to define the channel 214. At least one roller 222 or 224 of the pair of rollers (222, 224) is held in a biased position by a spring mechanism to be held against the flexible endoscope shaft or the flexible endoscope insertion component. This can be similar to FIGS. 5-8, for example.

In some embodiments, the manipulation assembly 210 can further include a drive assembly operatively connected to at least one roller of said pair of rollers (222, 224) to actively control the flexible endoscope shaft 216 or the flexible endoscope insertion component. See FIGS. 5-8 for an example of a drive assembly that includes an electric motor and a sprocket and serpentine chain configuration. However, the general concepts of the current invention are not limited to this example. For example, without limitation, direct drive electric motors may be used in some embodiments and/or other drive mechanism that do not use a serpentine chain configuration.

In this embodiment, the first end 204 of the support frame 202 is configured to be attached to the handpiece 206 of the flexible endoscope 208. In this embodiment, the support frame 202 is attached to a handheld manipulation robot 228.

FIG. 11 is a schematic illustration of an endoscope manipulation adapter 300 according to an embodiment of the current invention. The endoscope manipulation adapter 300 has a support frame 302 having a first end 304 configured to be rigidly fixed relative to a handpiece 306 of a flexible endoscope 308. The endoscope manipulation adapter 300 also has a manipulation assembly 310 arranged at a second end 312 of the support frame 302. The manipulation assembly 310 defines a channel 314 for at least one of a flexible endoscope shaft (not shown in FIG. 11) or a flexible endoscope insertion component 316 to traverse through in an axial direction 318 to assist in manipulating the at least one of the flexible endoscope shaft or the flexible endoscope insertion component 316 during use of the flexible endoscope 308.

The support frame 302 and the manipulation assembly 310 reserve space for accommodating slack 320 in the at least one of the flexible endoscope shaft or the flexible endoscope insertion component 316. The manipulation assembly 310 includes a pair of rollers (322, 324) arranged to define the channel 314. At least one roller 322 or 324 of the pair of rollers (322, 324) is held in a biased position by a spring mechanism to be held against the flexible endoscope shaft or the flexible endoscope insertion component (FIGS. 5-8, for example).

In some embodiments, the manipulation assembly 310 can further include a drive assembly operatively connected to at least one roller of the pair of rollers (322, 324) to actively control the flexible endoscope shaft or the flexible endoscope insertion component 316. See FIGS. 5-8 for an example of a drive assembly that includes an electric motor and a sprocket and serpentine chain configuration. However, the general concepts of the current invention are not limited to this example. Furthermore, manipulation assembly 310 can be a passive assembly in other embodiments of the current invention.

This embodiment can be used separately with a hand held endoscope and/or with a hand held or other robotic system according to other embodiments of the current invention. It can also be used in combination with any of the above-described embodiments such that both a flexible endoscope shaft and a flexible endoscope insertion component can be manipulated in the same system.

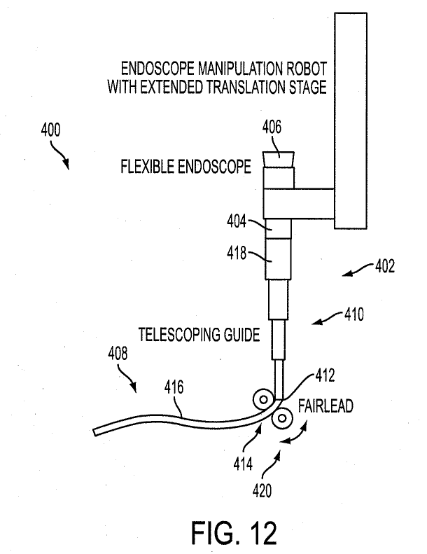

FIG. 12 is a schematic illustration of an endoscope manipulation adapter 400 according to an embodiment of the current invention. The endoscope manipulation adapter 400 has a support frame 402 having a first end 404 configured to be rigidly fixed relative to a handpiece 406 of a flexible endoscope 408. The endoscope manipulation adapter 400 also has a manipulation assembly 410 arranged at a second end 412 of the support frame 402. The manipulation assembly 410 defines a channel 414 for at least one of a flexible endoscope shaft 416 or a flexible endoscope insertion component (not shown in FIG. 12) to traverse through in an axial direction to assist in manipulating the at least one of the flexible endoscope shaft 416 or the flexible endoscope insertion component during use of the flexible endoscope 408.

In this embodiment, the manipulation assembly 410 can include telescoping tube 418 arranged to define at least a portion of the channel 414. The telescoping tube can be active through a drive mechanism, passive and/or hand operable. The manipulation assembly 410 can include a fairlead component 420 in some embodiments. Alternatively, instead of, or in addition to, fairlead component 420, an active assembly similar to the embodiments of FIGS. 9-11 could also be included.

This approach utilizes a constraint mechanism, such as a telescoping tube or compression spring and/or a fairlead mechanism to guide the scope direction. In this approach, the adapter could simply be a passive guide, relying on either robotic or human manipulation to provide all of the translational motion, or actively driven with its own motor(s). If the scope is mounted such that it is above the patient with the shaft feeding downward, held straight by gravity, then the telescoping tube could be omitted. This approach can provide several advantages over normal scope manipulation, including: the ability to move the scope without having to constrain it to enter the patient's orifice by hand, and the ability to enter the patient's orifice from any desired angle, thus allowing the scope and any other scope related mechanisms to be positioned to optimize surgical ergonomics.

In the passive case, this approach relies on external manipulation to provide translational motion, so the motion would be provided manually by the surgeon or robotically, possibly using a mechanism similar to an extended-length Robo-ELF.

In the active case, the fairlead could be a driven roller mechanism such as discussed above, except that it would be fixed with respect to the patient via the patient's mouthpiece, retractor, or other clinical equipment, rather than fixed with respect to the endoscope body. In this case, the surgeon would support the endoscope body, but the rollers would feed the scope shaft into the patient's orifice.

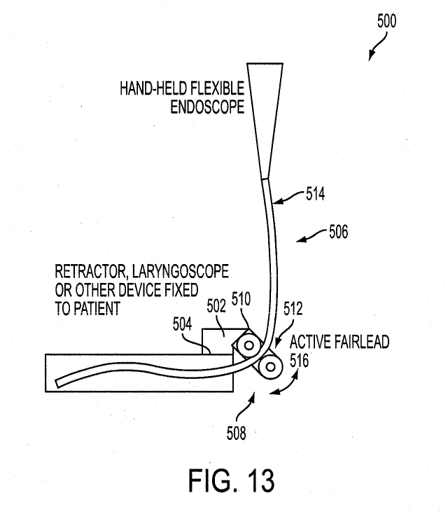

FIG. 13 is a schematic illustration of an endoscope manipulation adapter 500 according to an embodiment of the current invention. The endoscope manipulation adapter 500 includes a support frame 502 having a first end 504 configured to be rigidly fixed relative to a patient or an object to be viewed with a flexible endoscope 506. The endoscope manipulation adapter 500 also includes a manipulation assembly 508 arranged at a second end 510 of the support frame 502. The manipulation assembly 508 defines a channel 512 for a flexible endoscope shaft 514 to traverse through in an axial direction to assist in manipulating the flexible endoscope shaft 514 during use of the flexible endoscope 506. The manipulating assembly can include an active and/or passive fairlead 516, which can include active or passive rollers and/or an active or passive telescoping tube as described above.

In some embodiments, the first end of the support frame 502 can be configured to be attached to a surgical device 518 that is fixed relative to the patient during surgery. For example, the surgical device 518 could be at least one of a mouthpiece or retractor.

In some embodiments, endoscope manipulation adapters could be completely integrated with currently available robotic endolaryangeal systems and be controlled via the same motor controller so the surgeon would be able to operate both the current robotic system controls and the adapter controls using the same joystick mechanism. The endoscope manipulation adapter can also be miniaturized. The sprockets and the motor on the current design can be enclosed to allow for wash down cleaning in the operating room.

Apart from its application in a robotic endolaryngeal system, the endoscope manipulation adapter can be adapted for use with other scope manipulator devices not mentioned above that may or may not be robotically operated. In addition, although most of the above description is in the context of surgical applications, concepts of the current invention are not limited to only surgical uses. Other situations where endoscopes are used, such as for remote, dangerous and hazardous situations could benefit from embodiments of the current invention. He broad claims of the current invention are intended to include such applications.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. An endoscope manipulation adapter, comprising:
a support frame having a first end configured to be rigidly fixed relative to a handpiece of a flexible endoscope; and
a manipulation assembly arranged at a second end of said support frame,
wherein said manipulation assembly defines a channel for at least one of a flexible endoscope shaft or a flexible endoscope insertion component to traverse through in an axial direction to assist in manipulating said at least one of said flexible endoscope shaft or said flexible endoscope insertion component during use of said flexible endoscope.

2. An endoscope manipulation adapter according to claim 1, wherein said support frame and said manipulation assembly reserve space for accommodating slack in said at least one of said flexible endoscope shaft or said flexible endoscope insertion component.

3. An endoscope manipulation adapter according to claim 1, wherein said manipulation assembly comprises a pair of rollers arranged to define said channel.

4. An endoscope manipulation adapter according to claim 3, wherein at least one roller of said pair of rollers is held in a biased position by a spring mechanism to be held against said flexible endoscope shaft or said flexible endoscope insertion component.

5. An endoscope manipulation adapter according to claim 3, wherein said manipulation assembly further comprises a drive assembly operatively connected to at least one roller of said pair of rollers to actively control said flexible endoscope shaft or said flexible endoscope insertion component.

6. An endoscope manipulation adapter according to claim 1, wherein said manipulation assembly comprises a telescoping tube arranged to define said channel.

7. An endoscope manipulation adapter according to claim 1, wherein said first end of said support frame is configured to be attached to said handpiece of said flexible endoscope.

8. An endoscope manipulation adapter according to claim 1, wherein said first end of said support frame is configured to be attached to a robotic system that is attached to said handpiece of said flexible endoscope.

9. An endoscope manipulation adapter, comprising:
a support frame having a first end configured to be rigidly fixed relative to a patient or an object to be viewed with a flexible endoscope; and
a manipulation assembly arranged at a second end of said support frame,
wherein said manipulation assembly defines a channel for a flexible endoscope shaft to traverse through in an axial direction to assist in manipulating said flexible endoscope shaft during use of said flexible endoscope.

10. An endoscope manipulation adapter according to claim 9, wherein said support frame and said manipulation assembly reserve space for accommodating slack in said flexible endoscope shaft.

11. An endoscope manipulation adapter according to claim 9, wherein said manipulation assembly comprises a pair of rollers arranged to define said channel.

12. An endoscope manipulation adapter according to claim 11, wherein at least one roller of said pair of rollers is held in a biased position by a spring mechanism to be held against said flexible endoscope shaft.

13. An endoscope manipulation adapter according to claim 11, wherein said manipulation assembly further comprises a drive assembly operatively connected to at least one roller of said pair of rollers to actively control said flexible endoscope shaft.

14. An endoscope manipulation adapter according to claim 9, wherein said manipulation assembly comprises a telescoping tube arranged to define said channel.

15. An endoscope manipulation adapter according to claim 9, wherein said first end of said support frame is configured to be attached to a surgical device that is fixed relative to said patient during surgery.

16. An endoscope manipulation adapter according to claim 15, wherein said surgical device is at least one of a mouthpiece or retractor.
